(19) United States
(12) Patent Application Publication
(10) Pub. No.: US 2004/0229237 A1
(43) Pub. Date: Nov. 18, 2004

(54) Mast cell-specific signal transducer and cDNA thereof

(76) Inventor: Ryo Goitsuka, Tokyo (JP)

Correspondence Address: WENDEROTH, LIND & PONACK, L.L.P., 2033 K STREET N. W., SUITE 800, WASHINGTON, DC, 20006-1021, US

(21) Appl. No.: 10/717,619

(22) Filed: Nov. 21, 2003

Related U.S. Application Data
(63) Continuation-in-part of application No. 09/856,061, filed on Jul. 10, 2001.
Application No. 09/856,061 is a 371 of international application No. PCT/JP00/06351, filed on Sep. 18, 2000.

(30) Foreign Application Priority Data
Sep. 17, 1999 (JP) 1999-263778

(51) Int. Cl. C12Q 1/68; C07H 21/04; C07K 14/47; C07K 16/18

(52) U.S. Cl. 435/6; 435/69.1; 435/320.1; 435/325; 530/350; 530/388.1; 536/23.2

(57) ABSTRACT

The present invention provides a signal transducer specifically expressed in mouse mast cells that has the amino acid sequence of SEQ ID No. 2, a signal transducer specifically expressed in human mast cells that has the amino acid sequence of SEQ ID No. 4, polynucleotides encoding these proteins, an expression vector involving these polynucleotides, transformed cells induced by these expression vectors, and antibodies against the foregoing proteins. The signal transducer provided in the present invention is useful for screening of novel medicines against allergic diseases.

### TECHNICAL FIELD

The present invention relates to a signal transducer specifically expressed in mouse and human mast cell, and polynucleotides (cDNAs) encoding this protein molecule. More particularly, the present invention relates to a novel protein that is useful, for example, as a target molecule for screening a therapeutic agent for allergic diseases, and various genetic engineering materials useful for production and functional analysis of this protein.

### BACKGROUND ART

The type-I allergic response is a complicated immune reaction induced by release of granules containing histamine and serotonin through cross-linking of high affinity IgE receptors mainly expressed in the mast cell and basophilic leukocytes with IgE antibodies and allergens. This reaction has been elucidated to be composed of the following three stages:

A) An initial stage including production of cytokines such as IL-4 and IL-5 from T cell by stimulation of allergens, production of the IgE antibody from B cell, and differentiation and proliferation of the mast cells induced by production of the cytokines;

B) An intermediate stage from cross-linking of Fcε receptors by the IgE antibody and allergen to degranulation of the mast cell; and

C) A later stage such as enhanced vascular permeability by histamine and serotonin after degranulation.

The inventors of the present invention have isolated an adapter molecule BASH that is specifically expressed in B cell (J. Immunol., 161:5804-5808, 1998). This BASH has a similar molecular structure to SLP-76 (J. Biol. Chem., 270:7029-7032, 1995) that is expressed in T cell, and indicates the presence of a family of signal transducers specific to hemopoietic immunoreceptors through structural and functional analysis.

While suppression of IgE antibody production (Primary Stage) by B cell using a hyposentitization therapy, or suppression of the later stage by administration of anti-histaminic agent have been used today for treating allergies, neither of them serves as an effective therapy in the current situations.

A part of the molecular mechanism of the type-I allergy response is being made clear, on the other hand, as described above. However, the signal transduction mechanism involved in degranulation of mast cell through the high affinity IgE receptor has not been known yet. It is inevitable to elucidate the molecule involved in the degranulation process of mast cell not only for elucidating the molecular mechanism of the allergy response but also for developing therapeutic methods or therapeutic agents of the allergic diseases. Particularly, since the mast cell plays a critical role in expression of the allergic conditions, the signal transducer that is specifically expressed in mast cell is quite important for developing novel antiallergic agents that selectively block the Fcε receptor signal transduction system that causes the degranulation reaction involving release of histamine and serotonin.

The object of the present invention performed based on the foregoing situations is to provide signal transducers specifically expressed in mouse and human mast cells, and polynucleotides (cDNAs) encoding these protein molecules.

Another object of the present invention is to provide various genetic engineering materials involved in the signal transducers.

### DISCLOSURE OF INVENTION

For solving the problems above, the present invention provides the following inventions (1) to (10).

(1) A signal transducer specifically expressed in mouse mast cells, which is a purified protein having the amino acid sequence of SEQ ID No. 2.

(2) A signal transducer specifically expressed in human mast cells, which is a purified protein having the amino acid sequence of SEQ ID No. 4.

(3) A polynucleotide consisting of the base sequence of SEQ ID No. 1, which encodes the protein of (1).

(4) A polynucleotide having the base sequence of SEQ ID No. 3, which encodes the protein of (4).

(5) An expression vector involving the polynucleotide of (3).

(6) An expression vector involving the polynucleotide of (4).

(7) A cell transformed with the expression vector of (5), which produces the protein of (6).

(8) A cell transformed with the expression vector of (6), which produces the protein of (2).

(9) An antibody against the protein of (1).

(10) An antibody against the protein of (2).

### BRIEF DESCRIPTION OF THE DRAWINGS

FIG. 1 shows the results of Northern blot analysis investigating expression of MIST, BASH and SLP-76 in the hemopoietic and non-hemopoietic cell lines. 18-18: B-precursor cells, WEH11279: B cells, L1210: B-lymphocyte precursor cells, J558L and P3U1: plasma cells, EL-4 and BW5147: T cells, P388D1 and WEHI3: macrophages, P815: mast cell, B8/3: erythroblast, and B16,Y1, NIH3T3 and ES-E14: non-hemopoirtic cell lines.

FIG. 2 shows the results of RT-PCR analysis investigating expression of MIST in various hemopoietic cell lines.

FIGS. 3 and 4 show the results of immunohistological analysis investigating expression of MIST in inflammatory mast cell in atopic dermatitis of the NC/Nga mouse.

FIG. 5 shows the results of degranulation reaction of RBL-2H3 clone expressing wild-type or mutant MIST.

### BEST MODE FOR CARRYING OUT THE INVENTION

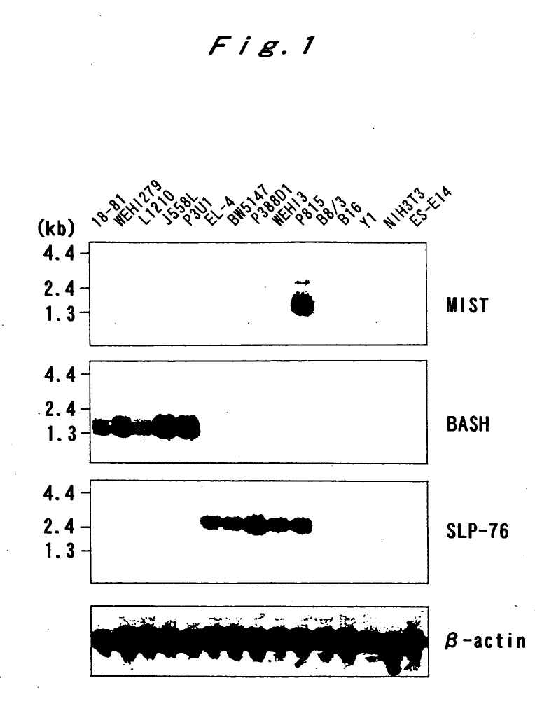

By screening the expression sequence tag (EST) database, the present inventor identified an EST clone from 13.5 day mouse embryo cDNA library (GenBank accession No. AA166259) which showed a significant amino acid homology to the SH2 domain of chicken BASH (J. Immunol. 161:5804-5808, 1998). The inventor further found that the 1.8 kb mRNA of this clone is not expressed in other hemopoietic cell lines and non-hemopoietic cell lines (such as B cell, T cell and macrophages) in which BASH and SLP-76 are expressed, and is expressed only in mastcytoma cell line P815 (FIG. 1). The expressed protein molecule was named as MIST (Mast Cell-specific Immunoreceptor Signal Transducer) form such specific expression pattern and its function to be described hereinafter.

Embodiments of the present invention will be described in detail hereinafter.

The MISTs according to the inventions (1) and (2) are proteins that are specifically expressed in mouse and human mast cells. The mouse MIST in the invention (1) is a protein encoded in the polynucleotide (full-length cDNA: SEQ ID No. 1) of the invention (3). The human MIST in the invention (2) is, on the other hand, a protein encoded in the polynucleotide (full-length cDNA: SEQ ID No. 3) of the invention (4).

While the mouse MIST in the invention (1) and human MIST in the invention (2) may be obtained by a method for isolating from organs and cell lines of mouse and human, respectively, by a method for preparing a peptide by a chemical synthesis based on the amino acid sequences provided by the present invention, or by a production method using a recombinant DNA technology using the polynucleotides of the inventions (3) and (4), the recombinant DNA method is preferably used. For example, RNA is prepared by in vitro transcription from a vector having the polynucleotides of the inventions (3) and (4), and MIST is expressed in vitro by in vitro translation using the RNA as a template. The mouse MIST and human MIST encoded by the polynucleotide can be expressed in large scale in prokaryotic cells such as E. coli and Bacillus subtilis, and in eukaryotic cells such as yeast, insect cells, mammal cells and plant cells by recombination of the coding region with the expression vector using a conventional method.

The polynucleotide (SEQ ID No. 1) of the invention (3) can be obtained by a chemical synthesis or screening of the mouse cDNA library. For cloning the desired polynucleotide from a cDNA library, an oligonucleotide is synthesized based on the base sequence in an arbitrary portion of SEQ ID No. 1, and the polynucleotide is screened by colony or plaque hybridization by the method known in the art using the oligonucleotide as a probe. Alternatively, oligonucleotides that can hybridize to both ends of the desired polynucleotide are synthesized, and the polynucleotide of the invention (3) is prepared by a PCR method using the oligonucleotides as primers and genomic DNA isolated from the mouse cells as a template.

The polynucleotide (SEQ ID No. 3) of the invention (4) also can be obtained by the same manner as SEQ ID No. 1 using human cDNa library etc.

For producing the MIST by expressing the polynucleotide in vitro translation, for example, the polynucleotide of the invention (3) or (4) is recombined into a vector having a RNA polymerase promoter [the inventions (5) and (6)], and the recombinant vector is added to an in vitro translation system such as a lysate of rabbit reticulocytes or wheat germ extract containing the RNA polymerase corresponding to the promoter, thereby producing the mouse and human MIST in vitro. Examples of the RNA polymerase promoters include T7, T3 and SP6. Examples of the vectors containing the RNA polymerase are pKA1, pCDM8, pT3/T7 18, pT7/3 19 and pBluescript II.

For producing the MIST by expressing the polynucleotide in microorganisms such as E. coli, an expression vector [the invention (5) and (6)] is prepared by recombining the polynucleotide of the invention (3) or (4) into an expression vector having an origin capable of replication in microorganisms, a promoter, a ribosome binding site, DNA cloning sites and terminator. After transforming host cell with this expression vector, the transformant obtained [the inventions (7) and (8)] is cultured for large scale production of MIST encoded by these polynucleotides in microorganisms. MIST fragments containing arbitrary regions may be obtained by adding an initiation codon and a termination codon before and after the arbitrary coding region. Or, the protein can be expressed as a fusion protein with other proteins. Only the protein regions encoded by this cDNA may be obtained by cleaving the fusion protein with an appropriate protease. Examples of the expression vector for use in E. coli include a pUC series vector, pBluescript II, pET expression system and pGEX expression system.

For producing the MIST by expressing the polynucleotide in eukaryotic cell, the polynucleotide of the invention (3) or (4) is recombined with an expression vector for eukaryotic cells that comprises a promoter, splicing site, poly(A) additional site to prepare a recombinamt vector [the inventions (5) and (6)], and the vector is introduced into the eukaryotic cell to transform a host cell [the inventions (7) and (8)]. Examples of the expression vectors include pKA1, pCDM8, pSVK3, pMSG, pSVL, pBK-CMV, pBK-RSV, EBV vector, pRS and pYES2. MIST may be expressed as a fusion protein to which various tags such as His tag, FLAG tag and GFP by using pIND/V5-His, pFLAG-CMV-2, pEGFP-N1 and pEGFP-C1 as an expression vector. While cultured cells of a mammal such as monkey kidney cells COS7 and Chinese hamster ovary cells CHO, budding yeast, dividing yeast, silkworm cells and African clawed frog egg cells are usually used as the eukaryotic cells, any eukaryotic cells may be used so long as they are able to express MIST. The expression vector can be introduced into the eukaryotic cell by a conventional method such as an electroporation method, a calcium phosphate method, a liposome method, and a DEAE dextran method.

A combination of separation methods known in the art may be used for purifying the desired protein from the culture after allowing MIST to express in the prokaryotic cells and eukaryotic cells. For example, these methods include treatment with a denaturation reagent such as urea or with a surface active agent, ultrasonic treatment, enzymatic digestion, salting-out and solvent precipitation method, dialysis, centrifugation, ultrafiltration, gel filtration, SDS-PAGE, isoelectric focusing electrophoresis, ion exchange chromatography, hydrophobic chromatography, affinity chromatography and reversed phase chromatography.

The mouse MIST of the invention (1) and the human MIST of the invention (2) contain any peptide fragments (five amino acid residues or more) represented by SEQ ID Nos. 2 and 4. These peptide fragments may be used for preparing antibodies. The MISTs of the inventions (1) and (2) are modified in any ways in the cell after translation. Accordingly, these modified proteins are also included within the scope of the present invention. Examples of modification after translation include elimination of N-terminal methionine, N-terminal acetylation, addition of sugar chains, restricted degradation by an intracellular protease, addition of miristoleic acid, isoprenylation and phosphorylation.

Polymorphism by individual differences is often observed in the animal gene. Accordingly, polynucleotides having addition or deletion of one or plural nucleotides and/or substitution with other nucleotides in the base sequence of SEQ ID Nos. 1 and 3 are also included within the scope of the present invention.

Likewise, MISTs having addition or deletion of one or plural amino acids and/or substitution with other amino acids caused by the alteration of polynucleotides as described above are also included within the scope of the present invention so long as it has an activity of the MIST containing the amino acid sequences of SEQ ID Nos. 2 and 4.

The polynucleotides in the inventions (3) and (4) also include DNA fragments (10 bp or more) comprising any partial base sequence of SEQ ID Nos. 1 and 3. DNA fragments comprising sense strand and antisense strans are also included within the scope as described above.

The antibodies according to the inventions (9) and (10) can be obtained from serums of an animals immunized with the proteins of the inventions (1) and (2). Chemically synthesized peptides based on the amino acid sequences of SEQ ID Nos. 2 and 4, and MIST itself expressed in the eukaryotic or prokaryotic cells may be used for the antigen. Otherwise, the antibodies may be produced from collected serums after introducing the expression vector for the eukaryotic cell into the muscle or skin of an animal by injection or using a gene gun (for example, the method described in Japanese Patent Publication No. 7-31387). The animals used include mouse, rat, rabbit, goat and chicken. Monoclonal antibodies against MIST can be obtained by preparing a hybridoma by fusing B cell extracted from an immunized animal with myeloma cells.

### EXAMPLES

The present invention is described in more detail with Examples, the present invention is not restricted in any sense by the Examples as set forth below.

### Example 1

### cDNA Cloning

Full-length mouse MIST cDNA was isolated from PT18 cDNA library with 5′- and 3′-RACE (Marathon cDNA amplification kit, made by Clontech Co.), using primers prepared based on the sequence information of EST clone (GenGank accession No. AA166259). The partial cDNA of human MIST was amplified by PCR using mRNA prepared from human cord blood mast cell (HCMC) cultured with IL-6 and the stem cell factor (SFC: Peprotech) according to the method in “Blood 86:3705-3714, 1995.

The sequence of the cDNA obtained was determined by the method known in the art, confirming that the mouse MIST cDNA comprises the base sequence represented by SEQ ID No. 1 and the human MIST partial cDNA comprises the base sequence represented by SEQ ID No. 3. It was also confirmed that the mouse MIST has the amino acid sequence represented by SEQ ID No. 2 with a molecular weight of about 60 kDa. Eight Tyr residues capable of phosphorylation are found in the mouse MIST from the N-terminus to the central part. The C-terminal part contains an SH2 domain which is most similar to the SH2 domain of mouse BASH and SLP-76 in amino acid level (41% and 53% identities, respectively). In addition, the central part of MIST is rich in Pro residues, and contains SH3 domain-binding motif. Consequently, MIST was confirmed to have the features as a signal molecule.

The human MIST showed, on the other hand, 60% homology with the mouse MIST in the amino acid level.

### Example 2

### Construction of Expression Vector

The coding region of the mouse MIST cDNA obtained in Example 1 was amplified by PCR, and the amplified region was inserted between the EcORI and Sal I sites of pCATneo expression vector (J. Immunol., 161:5804-5808, 1998) to construct a recombinant expression vector (pCATneo-MIST-WT).

The MIST mutant (MIST-YF) in which amino acids (Tyr) at 69, 96, 101, 153, 174 and 188 in SEQ ID No. 2 were substituted with other amino acids (Phe) was prepared by a PCR-based mutagenesis using a commercially available mutation kit (made by Stratagene Co.), and subcloned the MIST-YF into the pCATneo to construct a recombinant expression vector (pCATneo-MIST-YF).

### Example 3

### Preparation of Transformed Cells

The rat must cell line RBL-2H3 were transfected with the recombinant expression vectors pCATneo-MIST or pCATneo-MIST-YF prepared in Example 2 to prepare the transformed cell RBL-2H3-MIST and RBL-2H3-MIST-YE.

### Example 4

### Preparation of Antibody

An anti-MIST antibody was prepared from a rabbit immunized with a fusion protein of a polypeptide comprising the amino acid sequence 193-435 in SEQ ID No. 2 and glutathione-S-transferase (GST). The antisera were at first precleared with Seharose beads coupled with GST alone, and then purified with an affinity column coupled with GST-MIST fusion protein. Specificity of the antibody purified with affinity chromatography was confirmed by an immunoblot analysis on cell lysates from COS cells transfected with mouse MIST cDNA.

### Example 5

### Confirmation of MIST Expression in Various Cell Lines

Expression of the mouse and human MISTs obtained in Example 1 was confirmed by RT-PCR. The objective cells were IL-3-induced mouse bone marrow-derived mast cells (BMMC), mouse mast cell line PT18, human mast cells (HCNC) cultured with SCF and IL-6, and other hemocyte cell lines (Jurkat: human T cell, Romas: human B cell, KU812: human basophil precursor cell, EOL-1: human eosinophil precursor cell).

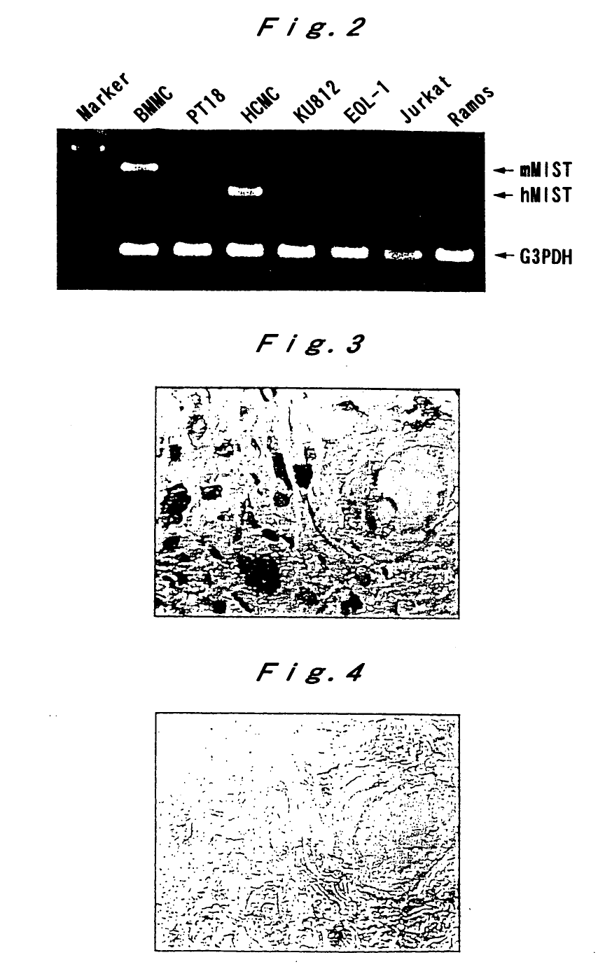

The results are as shown in FIG. 2. Although expression of MIST was found in mast cells BMMC, PT18 and HCNC, other cell lines showed no expression.

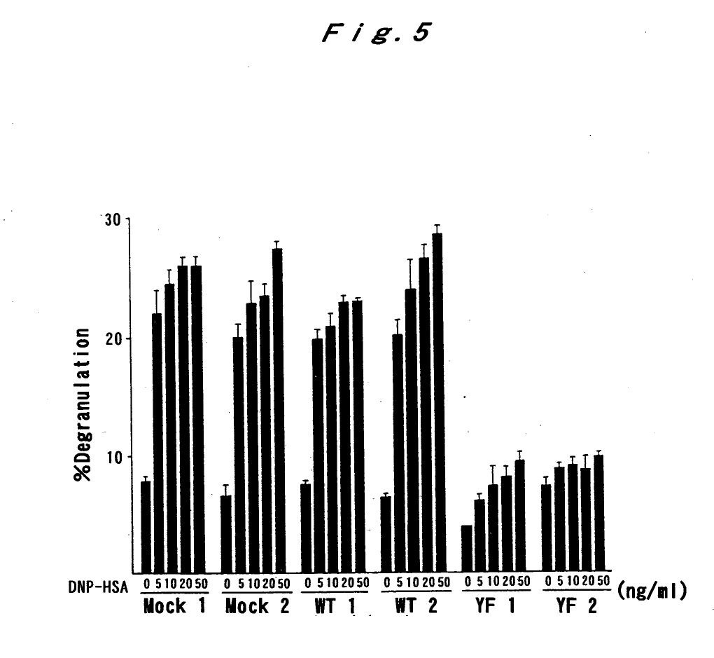

By using the anti-MIST antibody prepared in Example 4, serial tissue sections of NC/Nga mice, which spontaneously develop atopic dermatitis (J. Imunol., 9:461-466, 1997) were stained to clarify whether MIST protein is expressed in normal mast cells in in vivo. The results are shown in FIGS. 3 and 4. Expression of MIST was observed in the inflammatory mast cells in the mouse.

It was confirmed from the results as described above that MIST is a protein specifically expressed in mast cell.

### Example 6

### Confirmation of Phosphorylation of Tyrosine in MIST

Phosphorylation of tyrosine in MIST by stimulating with FcεRI was investigated using the rat mast cell line RBL-2H3 in which signal transduction of FcεR1 had been confirmed.

The transformed cell RBL-2H3-MIST prepared in Example 3 was cultured with 10 μg of anti-DNP mouse IgE (made by Sigma Co.) for 1 hour, and the cells were stimulated with 100 ng/ml of DNP-HSA. The cells were lysed with 1% NP40 lysis buffer, and the lysate was subjected to immune precipitation together with various antibodies.

Tyrosine of the MIST molecule was phosphorylated by stimulating the Fcε receptor on the mast cell IgE and antigens, and MIST associate with signal molecules such as PLC-γ and Vav. Consequently, the MIST molecule was confirmed to be a signal molecule existing at the downstream of the Fcε receptor. MIST was evidently phosphorylated by Lyn kinase among tyrosine kinases present in the mast cell, showing that the Lyn kinase has an important role for degranulation of the mast cell.

### Example 7

### Investigation of MIST Function in Degranulation of Mast Cell

The effect of over expression of MIST and mutation type MIST on degranulation of the cells was investigated using the transformed cells RBL-2H3-MIST and RBL-2H3-MIST-YF prepared in Example 3.

The cells were cultured with 1 pg/ml of anti-DNP mouse IgE overnight, washed twice with PBS, and stimulated with DNP-HSA at 37° C. for 30 minutes. Degranulation was confirmed by measuring release of β-hexosamimidase by the method described in the literature (Int. Immunol., 7:251-258, 1992).

The results are shown in FIG. 5. Although degranulation of the mast cell was not affected by stimulation with the Fcε receptor when a wild type MIST was over expressed, degranulation of the mast cell via the FCε receptor was significantly suppressed by over expression of the MIST mutant (MIST-YF).

It was confirmed from the results above that the MIST molecule plays an important role in the signal transduction pathway from stimulation by the Fcε receptor through degranulation.

### INDUSTRIAL APPLICABILITY

The present invention provides signal transducers that are specifically expressed in mouse and human mast cells, polynucleotides (cDNAs) encoding this protein molecule and various gene engineering materials concerning these signal transducers. Screening of novel agents for allergic diseases becomes possible by using these signal transducers as targets.

1. A signal transducer specifically expressed in mouse mast cells, which is a purified protein having the amino acid sequence of SEQ ID No. 2.

2. A signal transducer specifically expressed in human mast cells, which is a purified protein having the amino acid sequence of SEQ ID No. 4.

3. A polynucleotide consisting of the base sequence of SEQ ID No. 1, which encodes the protein of claim 1.

4. A polynucleotide having the base sequence of SEQ ID No. 3, which encodes the protein of claim 2.

5. An expression vector involving the polynucleotide of claim 3.

6. An expression vector involving the polynucleotide of claim 4.

7. A cell transformed with the expression vector of claim 5, which produces a protein having the amino acid sequence of SEQ ID No. 2.

8. A cell transformed with the expression vector of claim 6, which produces a protein having the amino acid sequence of SEQ ID No. 4.

9. An antibody against the protein of claim 1.

10. An antibody against the protein of claim 2.


SEQUENCE LISTING (4 sequences)

SEQ ID NO: 1 - DNA, 1721 bp, Mus musculus
  CDS (255)..(1562)
  ref <301>: AUTHORS: Goitsuka R., et al.
  ref <302>: TITLE: A BASH/SLP-76-related adaptor protein MIST/Clink involved in IgE receptor-mediated mast cell degranuation
  ref <303>: JOURNAL: Int. Immunol.
  ref <304>: VOLUME: 12
  ref <305>: ISSUE: 4
  ref <306>: PAGES: 573-580
  ref <307>: DATE: 2000
  ref <308>: DATABASE ACCESSION NUMBER: GenBank/AB021220
  ref <309>: DATABASE ENTRY DATE: 2000-05-26
acgaggccaaactgcccaggtctgtggctgcgtttctcggaaaaccaaaactcaacaggcacatacaaggcactctctgctgaaggactctgctgaggggagagaacatgtcaactctatcttacagagtgctccaggatgcgaccgtggaccccctttccaggagctagccgtctcaacactgagcccttgactaaaggaagactgagcaggctgagttgaagatccctctcttttgccaggtgccaaggaccatgaccagccagggcaataaaaggacaacgaaagaaggattcggtgatctgagattccagaacgtctctctgctgaaaaataggtcatggccaagcctcagcagtgccaaagggcggtgtcgagcggttctggaaccacttccggatcacagaaggaacttggctggggtcccaggtggagaaaaatgcaacagtaacaacgactacgaagatcctgagttccagctgctgaaggcatggccatcaatgaaaattttaccagccagacctatccaggaatcggaatacgcagatacacgctatttccaggatatgatggaggctccccttctgttacctcccaaggcttctgtctccactgagagacaaaccagggatgtgaggatgacacagctggaagaagtggacaagcctaccttcaaggatgtcagaagccaacgctttaaaggattcaaatacacaaaaataaacaagactcctttgccacctcctcggcctgctatcactctccccaagaagtaccaacccttacccccagcaccaccagaggagagcagtgcatacttcgctccaaagcccacctttccagaagtccagagggggcccaggcagaggagtgcaaaagacttcagtagggtccttggagcagaagaagaatctcaccaccagacaaagccagaatcttcttgcccatcatcaaaccaaaacacacagaagagtccacctgccattgccagctcttcctacatgccaggaaagcacagtatacaagccagagaccatacaggtagcatgcagcactgtcctgctcagagatgccaagctgcagccagccacagccctcgaatgctgccctatgaaaacacaaactcggagaaacctgaccccacaaagcctgatgagaaggatgtctggcagaatgaatggtacattggagaatacagtcgccaggcagtggaagatgtgttaatgaaagagaacaaggatggtacttttttggtccgagactgctctacaaaatccaaggcagaaccatatgttttggtggtgttttatgggaacaaggtctacaatgtgaaaatccgtttcctcgagagcaatcaacagtttgccctgggcacaggactacgaggaaatgagatgtttgattctgtggaagacatcattgaacactacacatattttcccattctgctaatagatgggaaagacaaggctgcacgcaggaaacagtgctacctcacccagccactgcctctcgccaggctccttctcactcagtactccagccaggcacttcatgagtaagaagcccagccagatatccccgcatcagtggcctgggccttgtctcattcctggctcaatggattcagttcttcttccatctgcatttatctgcaaagtattattttctgtgtcttcaagggatgattttttgactctgtaaaaaaaaaaaaaaaaaaa

SEQ ID NO: 2 - protein, 435 aa, Mus musculus
MTSQGNKRTTKEGFGDLRFQNVSLLKNRSWPSLSSAKGRCRAVLEPLPDHRRNLAGVPGGEKCNSNNDYEDPEFQLLKAWPSMKILPARPIQESEYADTRYFQDMMEAPLLLPPKASVSTERQTRDVRMTQLEEVDKPTFKDVRSQRFKGFKYTKINKTPLPPPRPAITLPKKYQPLPPAPPEESSAYFAPKPTFPEVQRGPRQRSAKDFSRVLGAEEESHHQTKPESSCPSSNQNTQKSPPAIASSSYMPGKHSIQARDHTGSMQHCPAQRCQAAASHSPRMLPYENTNSEKPDPTKPDEKDVWQNEWYIGEYSRQAVEDVLMKENKDGTFLVRDCSTKSKAEPYVLVVFYGNKVYNVKIRFLESNQQFALGTGLRGNEMFDSVEDIIEHYTYFPILLIDGKDKAARRKQCYLTQPLPLARLLLTQYSSQALHE

SEQ ID NO: 3 - DNA, 1814 bp, Homo sapiens
  CDS (241)..(1527)
  ref <308>: DATABASE ACCESSION NUMBER: GenBank/AB110420
  ref <309>: DATABASE ENTRY DATE: 2003-05-22
  ref <313>: RELEVANT RESIDUES: (1)..(1814)
ggctgcatttcacaggaaaccaagtctaaaacggacctatcaggaggttttctgctgaagggcactgcttagcatcgagaagaattcaacccaccgccttactaatttccagtgccccaaggtctctgcactgccgcccctcctcacaggagacggacacctcagcctagatcccttggtgctctccacgctgttcaggctgaattgaagagccctcttacccgccaggtgccaagaactatgaacaggcagggcaatagaaagacaactaaagaaggatccaacgatttgaaattccagaacttcagtctgccaaaaaacaggtcatggcctcgcatcaatagtgccacaggccagtaccagaggatgaacaagcctcttctagactgggaaagaaactttgctgcagtcctggatggagcaaaaggccacagtgatgatgactatgatgaccctgagcttcggatggaagagacatggcagtcgattaaaattttaccagcccggcctataaaggaatctgaatatgcagatacacactatttcaaggttgcaatggacactccccttccgttagacaccaggacctctatctccattggacagccgacctggaacacacagacgaggttggaaagagtggacaaacccatttccagggacgtcagaagccaaaacattaaaggagatgcatccgtaagaaagaacaagattcctttaccacctcctcggcctctcataacacttccgaagaagtaccaacccttgccccctgagccggagagcagcaggccacctttatctcagagacacacctttccagaagtccagggaatgcccagtcagataagcttaagggacttaagtgaggtccttgaagcagaaaaagttcctcataaccagaggaagcctgaatcaactcatctgttagaaaaccaaaatactcaagagattccacttgccattagcagttcttcattcacgacaagcaaccacagtgtgcaaaacagagatcatagaggaggcatgcagccctgttctcctcagagatgccagcctccagccagctgcagccctcacgaaaatatactgccctataaatacacaagctggagaccacctttccccaaaaggtctgatagaaaggatgtccagcacaatgaatggtacattggagaatacagccgccaggcagtggaagaggcattcatgaaggagaacaaggatggtagtttcttggtccgagattgttccacaaaatccaaggaagagccctatgttttggctgtgttttatgagaacaaagtctacaatgtaaaaatccgcttcctggagaggaatcagcagtttgccctggggacaggactcagaggagatgagaagtttgattcagtagaagacatcatcgaacactacaagaattttcccattatactaattgatgggaaagataaaactggggtccacaggaaacagtgtcacctcactcagccactccctctcaccagacacctcttgcctctgtagcctggtctttgtgttatctttggtttactggattcagcgcttccattgttttcattgatttcaaaagtttattttctgtgccttcaagggacaacttttttaactttggagaaaagaaaaacactctataacagagagtggaaaatcactcacggttttgaaagttcaaaccacagagaaaatatttataacatgcaaaaaataaaaacatttctagtaactggccactggaaaataaataaaaataaaaactaaaaaaaaagaaaaaaaaaaaaaaaaaaaaaaaa

SEQ ID NO: 4 - protein, 428 aa, Homo sapiens
MNRQGNRKTTKEGSNDLKFQNFSLPKNRSWPRINSATGQYQRMNKPLLDWERNFAAVLDGAKGHSDDDYDDPELRMEETWQSIKILPARPIKESEYADTHYFKVAMDTPLPLDTRTSISIGQPTWNTQTRLERVDKPISRDVRSQNIKGDASVRKNKIPLPPPRPLITLPKKYQPLPPEPESSRPPLSQRHTFPEVQGMPSQISLRDLSEVLEAEKVPHNQRKPESTHLLENQNTQEIPLAISSSSFTTSNHSVQNRDHRGGMQPCSPQRCQPPASCSPHENILPYKYTSWRPPFPKRSDRKDVQHNEWYIGEYSRQAVEEAFMKENKDGSFLVRDCSTKSKEEPYVLAVFYENKVYNVKIRFLERNQQFALGTGLRGDEKFDSVEDIIEHYKNFPIILIDGKDKTGVHRKQCHLTQPLPLTRHLLPL